(19) United States
(12) Patent Application Publication
(10) Pub. No.: US 2016/0317745 A1
(43) Pub. Date: Nov. 3, 2016

(54) Auto-Injection Device with Button Activation

(71) Applicant: NOVO NORDISK A/S, Bagsværd (DK)

(72) Inventors: Bastian Gaardsvig Kjeldsen, Hilleroed (DK); Brian Mouridsen, Fredensborg (DK)

(21) Appl. No.: 15/108,731

(22) PCT Filed: Jan. 5, 2015
(86) PCT No.: PCT/EP2015/050059

§ 371 Date: Jun. 28, 2016

(30) Foreign Application Priority Data
Jan. 3, 2014 (EP) 14150183.3

(51) Int. Cl. A61M 5/20; A61M 5/315

(52) U.S. Cl. CPC A61M 5/20; A61M 5/3155; A61M 5/31583; A61M 5/31535

(57) ABSTRACT

The present invention relates to an auto-injection device (100) repeatedly activateable for automatically expelling multiple set doses of a liquid drug. An activation button (190) protrudes from a proximal end of a housing (101) and is axially moveable between an activateable position and an activated position. The auto-injection device includes dose setting means comprising a dose setting device (170) that performs a helical movement away from a zero dose position during setting of a dose and returns by a helical movement to the zero dose position as the set dose is expelled. The dose setting device (170) is coupled to the activation button (190) to retain the activation button in the activated position when the dose setting device (170) assumes its zero dose position.

The invention relates to an auto-injection device for setting and expelling set doses of a liquid drug. In particular, the invention relates to a spring driven injection device of the type where an activation button is activated to trigger the device.

### BACKGROUND OF THE INVENTION

Medical drug delivery devices are used to deliver selected doses of medication to patients. Some medication, such as insulin, is self-administered. The typical diabetes patient will require injections of insulin several times during the course of the day.

State of the art drug delivery devices, such as the injection device disclosed in WO 01/95959, provides a user friendly and accurate device wherein most demands as regards patient needs are met. In such device the expelling mechanism is fully manual in which case a dose dial member and an injection button moves proximally during dose setting corresponding to the set dose size, and then is moved distally by the user to expel the set dose. Such devices are typically selectively operated in one of two different modes, i.e. a dose setting state and a dose expelling state. The dose dial member/the injection button may move slightly axially to toggle between the two operating states. A latch function may be incorporated serving to maintain the injection device in the dose expelling state during storage of the device.

An automatic injection device is disclosed in EP 338,806 wherein energy is stored in a spring during dose setting. A spring biased trigger slide is arranged to protrude from a side portion of the device. Subsequent to operating the trigger slide, the stored energy is released, thereby causing an expelling mechanism to drive a piston rod in an axial direction, thereby causing a set dose to be injected. More recent auto-injection devices typically employ an activation button arranged to protrude from a proximal end of the device housing. Examples of such auto-injection devices are disclosed in WO 06/076921 and WO 2008/116766.

Although auto-injection devices provide many advantages over the fully manual injection devices, the received user feedback during operation may be somewhat reduced. In a fully manual injection device the user may be able to perceive the progress of the injection simply by observing or by tactilely feeling the continued movement of the injection button whereas in an auto-injection device of the above-mentioned type the user is typically only provided an indication as to the completion of a full dose.

GB2443390 discloses a medicine delivery apparatus that includes an automatic driver and a user actuable trigger that is configured for being moved radially. A drum is rotatably and slideably mounted in the casing and includes a helical track adapted to engage with a cam follower disposed on the trigger. Prior to setting a dose the drum needs to be rotated before the cam follower of the trigger drops into the helical track. Further, WO 2009/097934 discloses an injection device that is configured for delivering a series of preset doses. A preactivation means is rotated in order to arm the device, where after an activating means in form of a pop-up button may be pressed in for activating medicament delivery.

Having regard to the above it is an object of the present invention to provide an improved auto-injection device for injection of multiple variably set doses, which is very simple to handle and which does not require any explanation to the user but wherein the working of the injection device is self-explanatory.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention relates to an auto-injection device for setting and expelling set doses of a liquid drug, comprising:
a housing defining a distal end and a proximal end, the housing adapted for receiving a cartridge so that liquid drug is expellable through a connected needle at the distal end of the housing,
dose setting means comprising a dose setting device configured for rotation by a helical movement relative to the housing,
an expelling mechanism comprising a spring drive configured for automatically expelling a set dose from a held cartridge upon activation,
an activation button activateable for enabling a set dose to be expelled, and
a release spring providing a force that urges the activation button towards the activateable position,
   wherein the auto-injection device defines a latch comprising a latch geometry coupled to the activation button and a cooperating retaining geometry coupled to the dose setting device, the latch being configured to retain the activation button in the activated position against the force of the release spring when the activation button has been activated,
   wherein the activation button protrudes from the proximal end of the housing and is axially moveable between an activateable position and an activated position, and the dose setting device is threadedly coupled relative to the housing so that it performs a helical movement away from a zero dose position during setting of a dose and returns by a helical movement to the zero dose position as the set dose is expelled,
   wherein:
   a) when the dose setting device assumes its zero dose position, the cooperating retaining geometry assumes a predefined axial position relative to the housing to enable cooperation between the cooperating retaining geometry and the latch geometry as the activation button assumes its activated position, and
   b) when the dose setting device assumes any other position than the zero dose position, the cooperating retaining geometry assumes an axial position which prevents cooperation between the cooperating retaining geometry and the latch geometry for retaining the activation button in its activated position.

In such configuration, until the auto-injection device is initially operated to set a dose, the activation button remains in its activated position. When the dose setting device is in its zero dose position, it makes no sense to push the activation button. As the activation button remains in its activated position, e.g. a pushed down position, it becomes clear for the user that the dose setting procedure is needed before the activation button is to be operated. This will add to the understanding of the intended use of the device. When dialling up a dose the activation button will be forced by the release spring and move into its activateable position, e.g. the activation button will pop out into an extended position. This signals that the device is loaded (a dose is set) and it is now possible to activate the activation button.

After the dose setting device has been operated to select the proper dose, the activation button may by manually pushed into its activated position enabling the expelling of the set dose. In the course of the expelling procedure, as the dose setting device returns to its zero dose position, the entire dose has been expelled and the latch will re-engage to retain the activation button in its activated position. Hence, the user receives feedback that the entire dose has been expelled.

In accordance with the above, the activation button is arranged to protrude from the proximal end of the housing. Subsequent to the size of a dose has been set by the user, the activation button may then, by application of an externally applied distal force, be moved from the activateable position (i.e. a proximal position) into its activated position (i.e. a distal position) in order to enable triggering of the expelling of a set dose. In particular embodiments, the activation button is fully embedded within the housing, or alternatively fully embedded within a manually operable part of the dose setting means, when the activation button is in the activated position. In such embodiments, as an additional advantage, the device will take up less space when stored, since the activation button does not extend from the housing of the device.

In some embodiments, the activation button directly operates and activates the expelling arrangement so that the spring drive mechanism automatically expels a set dose from the cartridge upon the activation button being manually moved into its activated position. In this way the activation button is provided as a release button for triggering the expelling operation.

The auto-injection device may in some embodiments comprise a needle shield for shielding an injection needle connected to the cartridge. The needle shield may be axially slideable relative to the injection needle and operable between an extended position and a retracted position. When the needle shield is in the extended position the user's skin is prevented from being penetrated by the injection needle whereas when the needle shield is in the retracted position the needle may pierce the skin of the user. In some embodiments the needle shield is coupled to the expelling arrangement so that upon the needle shield being shifted from the extended position to the retracted position the needle shield activates the expelling arrangement to expel the set dose, i.e. the needle shield forms a trigger element. In such an embodiment, the activation button arranged at the proximal end of the housing may be operably coupled to the needle shield in such a way that the needle shield, when in the extended position, will be prevented from moving into the retracted position as long as the activation button is in the activateable position. Subsequent to the dose dial member has been operated and the activation button has been released into its activateable position, the needle shield is allowed to be moved into its retracted position thereby enabling activation of the expelling arrangement for automatically expelling the set dose. Hence, the auto-injection device is configured as a needle shield triggered device which is manually triggered by pressing the needle shield against an injection site. However, the device may be so configured that the needle shield triggering is only possible after firstly a dose has been set by moving the dose setting device away from the zero dose position and secondly the activation button has been moved into its activated position. In such an embodiment, the activation button serves as a safety release member which is required to be operated before needle shield triggering is enabled.

In the context of the present invention, the definition that the dose setting device is configured for rotation by a helical movement relative to the housing means that the dose setting device and an associated housing component are mutually connected so as to allow relative rotational movement there between. Said housing component may define a window component that is positioned adjacent the dose setting device so that dose indicia disposed on the dose setting device is visually observable in a window formed by the window component. During dose setting as well as during dose expelling relative helical rotational movement occurs between the dose setting device and the window component. In some embodiments, the dose setting device rotates relative to the window component. In other embodiments, the window component rotates relative to the dose setting device. In still other embodiments, during dose setting, a first component selected from one of the dose setting device and the window component rotates relative to the other component whereas during dose expelling said “other component” rotates relative to the first component. All said situations are construed to be encompassed by the accompanying claims when referring to the dose setting device performing a helical movement.

In the context of the present invention, the definition “the dose setting device is threadedly coupled relative to the housing so that it performs a helical movement away from a zero dose position during setting of a dose” means that the dose setting device (or the window component) performs a helical movement away from the zero dose position as the dose is being dialed up during the setting of a dose. In some embodiments, the auto-injection device may be so configured that an initially dialed dose may be decreased by dialing down the dose setting device so that it performs a helical movement towards the zero dose position without liquid drug being expelled from the auto-injection device.

In accordance with such configuration the dose setting device and the housing experiences relative axial movement as the dose setting device is rotated. Generally, when the dose setting device is rotated the dose setting device moves axially relative to the activation button. Respective cooperating latch features associated with the dose setting device and the activation button may be arranged so that they are positioned relative to each other for the latch to become engaged only when the dose setting device assumes a predefined position or, alternatively, assumes one of a subset of predefined positionable positions.

In some embodiments, when the activation button assumes its activated position retained by cooperation between the latch geometry and the cooperating retaining geometry, rotation of the dose setting device away from its zero dose position releases the latch so that the activation button is automatically moved into the activateable position urged by the force for the release spring.

In some embodiments, the latch comprises one or more sets of cooperating latch features, each set of cooperating latch features comprising a latch geometry cooperating with a cooperating retaining geometry.

In further embodiments, the auto-injection device is configured for multi-use operation and thus being repeatedly operable for setting and expelling a multitude of individual administered doses.

Subsequent to the expelling of the entire amount of an individually set dose, the activation button is retained in the activated position until the dose setting device is operated and dialed away from its zero dose position again. This is enabled by the specific movement of the dose setting device during the dose setting procedure and during the dose expelling procedure relative to the position of the activation button.

The at least one of said sets of cooperating latch features may comprise a) a latch geometry disposed on the activation button, and b) a cooperating retaining geometry disposed on the dose setting device. Each of the said sets of latch geometries and the cooperating retaining geometries may be so configured that, when the dose setting device assumes its zero dose position and when the activation button assumes its activated position, the latch geometries engage the cooperating retaining geometries to retain the activation button in the activated position.

The auto-injector may be so configured that when the dose setting device assumes any other position than the zero dose position, the cooperating retaining geometry is axially spaced away from the corresponding latch geometry preventing the latch from retaining the activation button in the activated position.

Due to the dose setting device performing a helical movement away from a zero dose position during setting of a dose and performing a helical movement as the set dose is expelled the relative axial position between the latch geometry and the cooperating retaining geometry is altered allowing the latch to become released upon dialing away from the zero dose position and only to become latched again when both conditions for latching occurs: i.e. a) the dose setting device assuming its zero dose position and b) the activation button assuming its activated position.

The dose setting device may be coupled relative to other structures in the device by a threaded connection, such as a permanent threaded engagement. The threaded connection may in some embodiments be provided as a thread exhibiting a constant lead. In other embodiments the lead of the threaded connection may be provided as one or more threaded segments exhibiting a variable lead along the extension of the thread. In some embodiments, the dose setting device is threadedly engaging a window component of the device so that the dose setting device is moved axially relative to the window component as the dose setting device is rotated. Said window component may be attached to the housing of the device or, alternatively, formed unitarily with a component forming the housing. The threaded connection may be arranged for permanent engagement. Hence, the threaded engagement is maintained during setting of a dose as well as during expelling of a set dose.

The dose setting device may be configured to be positionable relative to the housing in a plurality of angular rest positions. Hence, the dose setting device may be configured for being manipulated so as to be rotated back and forth between a range of discrete rotational rest positions. Said plurality of angular rest positions may be evenly distributed between the zero dose position and a maximum settable dose position.

In certain embodiments the dose setting device is rotatable relative to the housing by a rotational movement exceeding 360 degrees. Thus, in some embodiments the dose setting device may be configured as a multi-turn operable dose setting device.

The dose setting device may in some embodiments be formed as a scale drum having a series of dose indications arranged thereon along a helical path. In other embodiments, the dose setting device is not necessarily provided as a scale drum but rather provided as a component that has no scale indications, such as a component that is hidden within the housing and not viewable from outside the housing. The dose setting device is preferably a component that relative to a secondary component moves axially away from a zero dose position as a dose is dialed up and which returns to the zero dose position as a dose is being expelled. In accordance with certain embodiments, this component may be rotated relative to the secondary component at least when the component is moved during dose setting.

The dose setting means may comprise a manually operable dose dial member arranged for being rotated relative to the housing or relative to said window component in order to set the size of a dose to be subsequently expelled. The dose setting device may be coupled with the dose dial member in a way so that the dose setting device is rotated as the dose dial member is manually rotated. In some embodiments, the dose dial member is axially fixed relative to the housing. In such embodiments, the dose setting device may be so coupled to the dose dial member so that relative rotation is prevented and so that relative axial movement is allowed. In still other embodiments, the dose setting device at least partly forms the dose dial member so that the dose setting device defines an element that is manually gripable by the user and rotatable relative to the housing in order to set a dose. In some embodiments, the amount of rotation that the dose setting device is rotated away from a zero dose position will be proportional to size of the set dose.

The latch may be so configured that when the dose setting device assumes any other position than the zero dose position the latch does not retain the activation button when positioned in the activated position and the release spring acts to move the activation button towards its activateable position. However, in some embodiments of the auto-injection device being designed to provide discrete rest positions for the dose setting device, when the dose setting device is positioned in intermediate positions between the rest position corresponding to the zero dose position and the first consecutive rest position, i.e. the rest position neighboring the zero dose position, the latch may be configured to retain the activation button in the activated position. In such embodiments, the latch is so configured that when the dose setting device assumes rotational positions ranging from said first consecutive rest position to the maximum dose position the latch does not retain the activation button when the activation button assumes its activated position and the release spring acts to move the activation button towards its activateable position.

In some embodiments the latch geometry comprises a hook member and the cooperating retaining geometry comprises a track arranged to receive the hook member as the dose setting device is rotated towards its zero dose position to thereby retain the hook member against axial displacement.

Such configuration may for example exhibit an L-shaped track adapted to receive and retain a portion of the hook member which may include an enlarged head for insertion into the track. In other embodiments the track instead forms an indentation adapted to receive a protruding part of the hook member.

In alternative embodiments the latch geometry comprises a snap member and the cooperating retaining geometry comprises a snap retainer wherein the snap member is releasably received and retained by the snap retainer when the activation button and the dose setting device assume a pre-defined relative orientation.

In some embodiments the auto-injection device includes one or more snap members that each is provided as a snap arm that engages a corresponding snap retainer.

Other embodiments include an auto-injection device wherein one of the activation button and the dose setting device include a cylindrical face, wherein the snap retainer comprises a circumferentially extending ledge arranged on said cylindrical face and wherein the snap member is provided as a snap arm configured to engage and snap behind said circumferential extending ledge when the activation button assumes its activated position while the dose setting device assumes its zero dose position.

The snap member may be configured for being released from the snap retainer upon the dose setting device moving relatively to the activation button as the dose setting device is rotated away from the zero dose position.

Hence, when the user exerts a torque on the dose setting device, e.g. by manually rotating a dose dial member away from the zero dose position, upon exertion of a torque beyond a predefined threshold, the snap member or snap arm is released from the snap retainer and the activation button is automatically moved to the activateable position caused by the force provided by the release spring.

In some embodiments, the snap member or snap arm snaps free from the snap retainer when the dose setting device is moved axially relative to the activation button upon the dose setting device being rotated away from the zero dose position. In such embodiments, the activation button may or may not be rotationally fixed relative to the housing. In other embodiments, the snap member or snap arm snaps free from the snap retainer as the dose setting device is moved rotationally relative to the activation button upon the dose setting device being rotated away from the zero dose position. In such embodiments, the activation button is preferably rotationally fixed relative to the housing.

The latch geometry may comprise a flexible member, the flexible member being able to flex when the activation button is moved from its activateable position into the activated position while the dose setting device maintains its zero dose position. Hence, the flexible member serves to allow the latch geometry to engage the cooperating retaining geometry.

In such embodiment, after a dose setting procedure has been initiated and the activation button has been released into its activateable position, the activation button may be brought into its activated position again although no expelling following the previous dose setting procedure has been performed. Such situation might occur if a user by mistake dials up a dose followed by the user dialing back the dose setting device to its zero dose position. This will typically leave the activation button in its activateable position. But the flexible member allows the latch geometry to engage the cooperating retaining geometry to establish the retainment of the activation button simply by manually forcing the activation button into its activated position by applying a distal force on the activation button. Both for the embodiments incorporating a hook and track configuration as well as in embodiments incorporating the said snap members and snap retainers such function will be obtainable.

In certain embodiments one of the latch geometry and the cooperating retaining geometry comprise an inclined cam while the other of the latch geometry and the cooperating retaining geometry comprise a cam follower for engaging the inclined cam. The inclined cam and the cam follower may be so configured that, when no externally applied distal force is applied to the activation button and the activation button assumes the activateable position, the inclined cam and cam follower acts to move the activation button into its activated position as the dose setting device is returned to the zero dose position. The term “inclined cam” is construed to mean that the cam extends at an inclined angle relative to the axis along which the release button is moved. For a pen-formed device such axis is typically a central axis along which a piston of the cartridge is moved during expelling of a set dose.

In further embodiments the latch geometry and the cooperating retaining geometry are configured for generating a click sound as the latch geometry engages the cooperating retaining geometry upon the dose setting device being returned to its zero dose position. Hence an end of dose click may be provided by means of the latch function signaling the completion of the expelling of a set dose.

The spring drive of the auto-injection device may comprise a drive element configured for distally moving a piston of a held cartridge and a spring element coupled to the drive element. The spring element may be configured for holding stored energy and, upon activation, releasing energy for urging the drive element to drive the piston for expelling a set dose during an expelling procedure. In some embodiments a piston rod is arranged to interconnect the piston of a held cartridge and the drive element.

The expelling mechanism may further comprise a clutch element coupled to the activation button and the drive element to control release of stored energy of the spring element. When the activation button is in the activated position the clutch element enables release of stored energy, and when the activation button is in the activateable position the clutch element prevents release of stored energy.

In some embodiments of the auto-injection device, the device may be so configured that, in the course of an expelling procedure, upon removal of an initially applied external distal force on the activation button, the force of the release spring acts to move the activation button to the activateable position to interrupt the expelling procedure. Upon renewed application of an external distal force on the activation button, expelling of the remaining part of the interrupted dose administration will be resumed.

In a second aspect, the invention relates to an auto-injection device for setting and expelling set doses of a liquid drug, comprising:
a housing defining a distal end and a proximal end, the housing adapted for receiving a cartridge so that liquid drug is expellable through a connected needle at the distal end of the housing,
dose setting means comprising a dose setting device that moves axially in the housing, the dose setting device configured to be moved axially in a first direction away from a zero dose position during setting of a dose and to move axially in a direction counter to the first direction and return to the zero dose position as the set dose is expelled,
an expelling arrangement comprising a spring drive mechanism that automatically expels a set dose from the cartridge upon activation,
an activation button arranged at the proximal end of the housing and activateable for enabling a set dose to be expelled, the activation button being axially moveable between an activateable position and an activated position, and
a release spring providing a force that urges the activation button towards the activateable position.

The auto-injection device defines a latch configured to retain the activation button in the activated position against the force of the release spring when the activation button has been activated, said latch being configured to retain the activation button in the activated position when the dose setting device assumes its zero dose position and configured not to retain the activation button in the activated position when the dose setting device assumes a position other than the zero dose position so that the activation button is automatically moved to the activateable position as the dose setting device is moved axially away from its zero dose position.

In a third aspect, the invention relates to an auto-injection device for setting and expelling set doses of a liquid drug, comprising:
a housing defining a distal end and a proximal end, the housing adapted for receiving a cartridge so that liquid drug is expellable through a connected needle at the distal end of the housing,
dose setting means comprising a dose setting device rotatably arranged in the housing, the dose setting device configured to perform a helical movement away from a zero dose position during setting of a dose and to return by a helical movement to the zero dose position as the set dose is expelled,
an expelling arrangement comprising a spring drive mechanism that automatically expels a set dose from the cartridge upon activation,
an activation button arranged at the proximal end of the housing and activateable for enabling a set dose to be expelled, the activation button being axially moveable between an activateable position and an activated position, and
a release spring providing a force that urges the activation button towards the activateable position.

The auto-injection device defines a latch configured to retain the activation button in the activated position against the force of the release spring when the activation button has been activated, said latch being configured to retain the activation button in the activated position when the dose setting device assumes its zero dose position and configured not to retain the activation button in the activated position when the dose setting device assumes a position other than the zero dose position so that the activation button is automatically moved to the activateable position as the dose setting device is rotated away from its zero dose position.

The respective devices of the second and the third aspect may include any of the features or combination of features as defined above in connection with the first aspect.

### DEFINITIONS

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” or “Needle” is used to describe the actual conduit performing the penetration of the skin during injection. A needle cannula is usually made from a metallic material such as e.g. stainless steel and connected to a hub to form a complete injection needle also often referred to as a “needle assembly” or simply an “injection needle” A needle cannula could however also be made from a polymeric material or a glass material. The hub also carries the connecting means for connecting the needle assembly to an injection apparatus and is usually moulded from a suitable thermoplastic material. The “connection means” could as examples be a luer coupling, a bayonet coupling, a threaded connection or any combination thereof e.g. a combination as described in EP 1,536,854.

Needle assemblies specially designed for pen injections systems are defined in ISO standard No. 11608, part 2, and are often referred to as “pen needles”. Pen needles have a front-end for penetrating into the skin of the user and a back-end for penetrating into the cartridge containing the drug.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing. When reference is made to a “zero dose position” of the scale drum, this does not necessarily mean that the number “0” is present, however it merely refers to the position of the scale drum in which no dose has been set.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the back-end of an injection needle. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower neck portion into which the rubber plunger cannot be moved, not all of the drug contained inside the cartridge can be expelled. The term “initial quantum” therefore refers to the initial quantum of the injectable content. The term “remaining content” in the same way refers to the remaining injectable content.

Using the term “Automatic” in conjunction with injection device means that, the injection device is able to perform the injection without requiring the user of the injection device to manually deliver the force needed to expel the drug. The force is typically delivered by a spring device, such as a gas spring or as herein described by a spring element that is strained by the user during dose setting. Such springs are usually prestrained in order to avoid problems of delivering very small doses. Alternatively, the spring can be preloaded by the manufacturer with a preload sufficient to empty the drug cartridge though a number of doses. Typically the user activates a latch or a button on the injection device to release the force accumulated in the spring when carrying out the injection. In the following, the term “Auto-injection device” is in addition used for such automatic injection devices.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

In the following the invention will be further described with references to the drawings, wherein:

FIG. 1 shows a perspective view of a prior art pen device,

FIG. 2 shows in an exploded view the components of the pen device of FIG. 1,

FIGS. 3A and 3B show in sectional views an expelling mechanism of the pen device of FIG. 1 in two states,

FIGS. 3C-3E show components of the pen device of FIG. 1,

FIG. 4A shows a perspective view of operable elements of a first embodiment of an auto-injection device in accordance with the present invention where a dose has been set,

FIG. 4B shows a perspective view of operable elements of the first embodiment shown in FIG. 4A but after dose expelling,

FIG. 5A shows a partly cut perspective view of the auto-injection device of FIG. 4A where a dose has been set,

FIG. 5B shows a partly cut perspective view of the auto-injection device of FIG. 4B where a dose has been expelled,

FIG. 5C shows a detailed perspective view of a dose setting device of the auto-injection device of FIG. 4A,

FIG. 6 shows a perspective view of a dose setting device and an activation button of the auto-injection device shown in FIG. 4A,

FIG. 7 shows a partly cut side view of the auto-injection device shown in FIG. 4A,

FIGS. 8A and 8B show in sectional views an expelling mechanism of a second embodiment of an auto-injection device in accordance with the present invention, where FIG. 8A shows the device where a dose has been set and FIG. 8B shows the device where a dose has been expelled,

FIGS. 9A and 9B show in sectional views an expelling mechanism of a third embodiment of an auto-injection device in accordance with the present invention, where FIG. 9A shows the device where a dose has been set and FIG. 9B shows the device where a dose has been expelted, and

FIG. 10 shows a schematic view of a dose setting device and an activation button of the auto-injection device shown in FIG. 9A.

The figures are schematic and simplified for clarity, and they just show details which are essential to the understanding of the invention, while other details are left out. Throughout, the same reference numerals are used for identical or corresponding parts.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. In that context it may be convenient to define that the term “distal end” in the appended figures is meant to refer to the end of the injection device which usually carries the injection needle whereas the term “proximal end” is meant to refer to the opposite end pointing away from the injection needle and carrying the dose dial member and the activation button as depicted in the figures.

When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. When it is defined that members are mounted axially free to each other it generally indicates that they can be moved relative to each other, typically between defined stop positions whereas when it is defined that members are mounted rotationally free to each other it generally indicates that they can be rotated relative to each other either freely or between defined stop positions. The terms “assembly” and “subassembly” do not imply that the described components necessary can be assembled to provide a unitary or functional assembly or subassembly during a given assembly procedure but is merely used to describe components grouped together as being functionally more closely related.

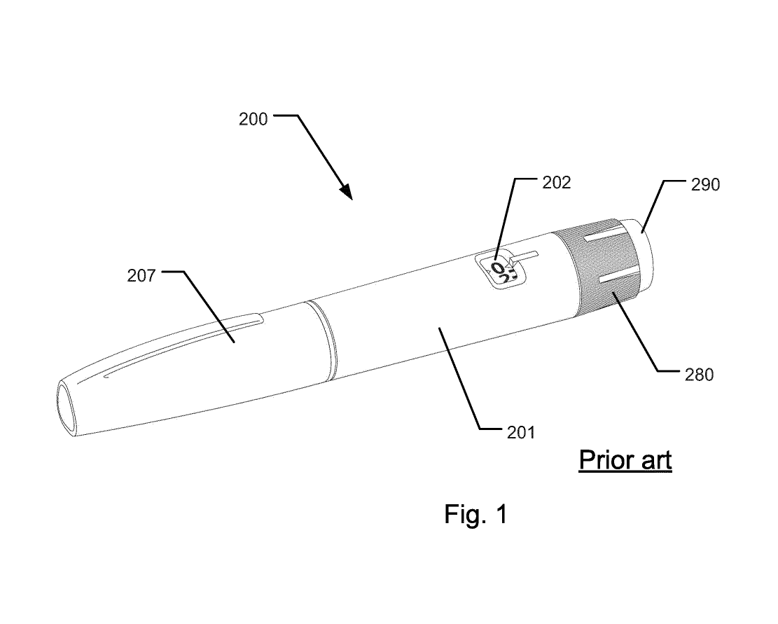

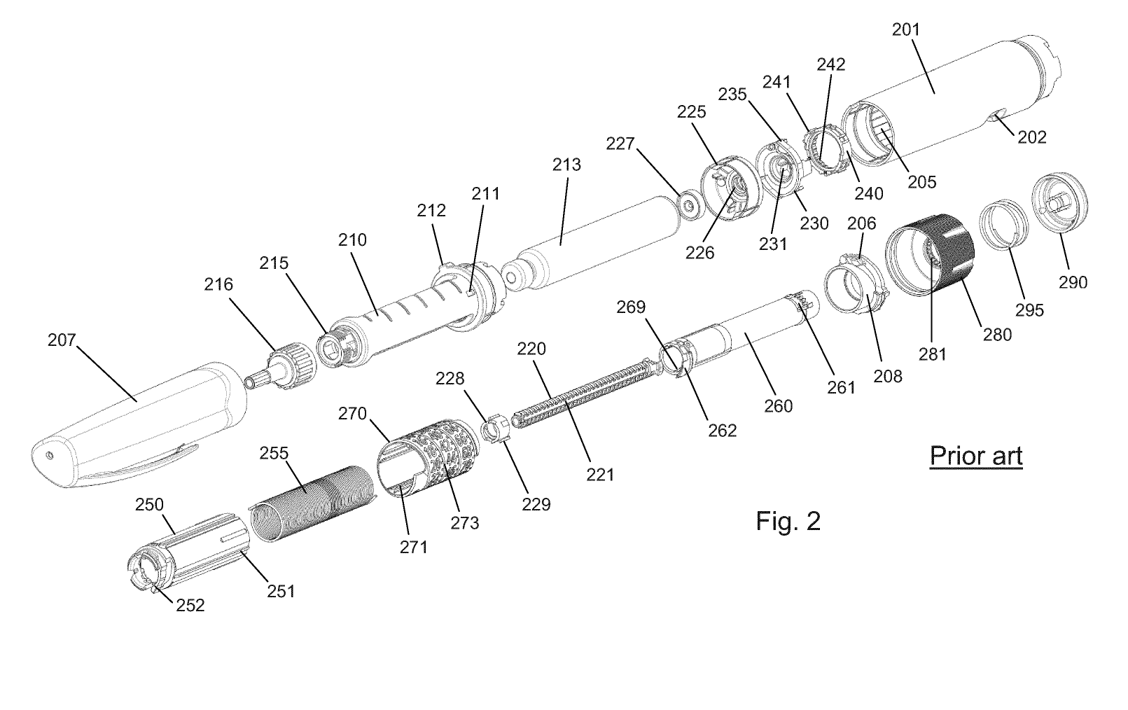

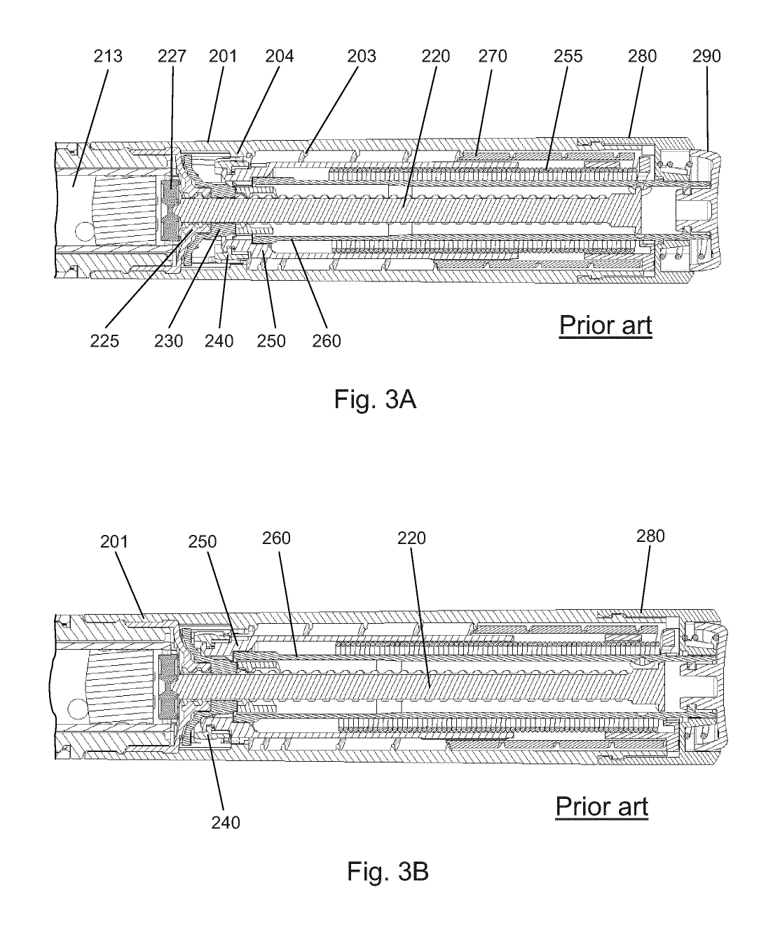

FIG. 1 shows a prior art drug delivery device in the form of a pen-formed auto-injection device 200, i.e. a so-called “injection pen” that includes an expelling mechanism incorporating a spring drive. FIG. 2 shows an exploded view of the prior art auto-injection device 200 shown in FIG. 1. FIGS. 3A and 3B show cross sectional views of the expelling mechanism of the prior art auto-injection device 200 shown in FIGS. 1 and 2 where FIG. 3A shows the device in dose setting state and FIG. 3B shows the device in dose expelling state.

In the present context the device 200 represents a “generic” drug delivery device providing a specific example of a device which, in accordance with the present invention, may be modified in order to obtain a device that provides improved user feedback. As the invention relates to elements of a device which mainly pertains to user feedback, an exemplary embodiment of such a device will be described for better understanding of the invention.

The pen device 200 comprises a cap part 207 and a main part having a proximal body or drive assembly portion with a housing 201 in which a drug expelling mechanism is arranged or integrated, and a distal cartridge holder portion in which a drug-filled transparent cartridge 213 with a distal needle-penetrable septum is arranged and retained in place by a non-removable cartridge holder attached to the proximal portion, the cartridge holder having openings allowing a portion of the cartridge to be inspected as well as distal coupling means 215 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose dial member 280 serves to manually set a desired dose of drug shown in display window 202 and which can then be expelled when the release button 290 is actuated. Depending on the type of expelling mechanism embodied in the drug delivery device, the expelling mechanism may comprise a spring as in the shown embodiment which is strained during dose setting and then released to drive the piston rod when the release button 290 is actuated.

As appears, FIG. 1 shows a drug delivery device of the pre-filled type, i.e. it is supplied with a pre-mounted cartridge and is to be discarded when the cartridge has been emptied. In alternative embodiments, and in accordance with the present invention, the drug delivery device may be designed to allow a loaded cartridge to be replaced, e.g. in the form of a “rear-loaded” drug delivery device in which the cartridge holder is adapted to be removed from the device main portion, or alternatively in the form of a “front-loaded” device in which a cartridge is inserted through a distal opening in the cartridge holder which is non-removable attached to the main part of the device.

More specifically, referring to FIG. 2, the pen comprises a tubular housing 201 with a window opening 202 and onto which a cartridge holder 210 is fixedly mounted, a drug-filled cartridge 213 being arranged in the cartridge holder. The cartridge holder is provided with distal coupling means 215 allowing a needle assembly 216 to be releasably mounted, proximal coupling means in the form of two opposed protrusions 211 allowing a cap 207 to be releasably mounted covering the cartridge holder and a mounted needle assembly, as well as a protrusion 212 preventing the pen from rolling on e.g. a table top. In the housing distal end a nut element 225 is fixedly mounted, the nut element comprising a central threaded bore 226, and in the housing proximal end a spring base member 208 with a central opening is fixedly mounted. A drive system comprises a threaded piston rod 220 having two opposed longitudinal grooves and being received in the nut element threaded bore, a ring-formed piston rod drive element 230 rotationally arranged in the housing, and a ring-formed clutch element 240 which is in rotational engagement with the drive element (see below), the engagement allowing axial movement of the clutch element. The clutch element is provided with outer spline elements 241 adapted to engage corresponding splines on the housing inner surface, this allowing the clutch element to be moved between a rotationally locked proximal position, in which the splines are in engagement, and a rotationally free distal position in which the splines are out of engagement. As just mentioned, in both positions the clutch element 240 is rotationally locked to the drive element 230. The drive element comprises a central bore with two opposed protrusions 231 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 235 adapted to engage corresponding ratchet teeth 205 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3C and 3D.

On the piston rod an end-of-content (EOC) member 228 is threadedly mounted and on the distal end a washer 227 is rotationally mounted. The EOC member comprises a pair of opposed radial projections 229 for engagement with the reset tube (see below).

The dial system comprises a ratchet tube 250, a reset tube 260, a scale drum 270 with an outer helically arranged row of dose numerals, a user-operated dose dial member 280 for setting a dose of drug to be expelled, a release button 290 and a torque spring 255 (see FIGS. 3A and 3B). The reset tube is mounted axially locked inside the ratchet tube but is allowed to rotate a few degrees (see below). The reset tube comprises on its inner surface two opposed longitudinal grooves 269 adapted to engage the radial projections 229 of the EOC member, whereby the EOC can be rotated by the reset tube but is allowed to move axially. The clutch element is mounted axially locked on the outer distal end portion of the ratchet tube 250, this providing that the ratchet tube can be moved axially in and out of rotational engagement with the housing via the clutch element. The dose dial member 280 is mounted axially locked but rotationally free on the housing proximal end, the dose dial member being under normal operation rotationally locked to the reset tube (see below), whereby rotation of dose dial member results in a corresponding rotation of the reset tube and thereby the ratchet tube. The release button 290 is axially locked to the reset tube but is free to rotate. A return spring 295 provides a proximally directed force on the button and the thereto mounted reset tube. The scale drum 270 is arranged in the circumferential space between the ratchet tube and the housing, the drum being rotationally locked to the ratchet tube via cooperating longitudinal splines 251, 271 and being in rotational threaded engagement with the inner surface of the housing via cooperating thread structures 203, 273, whereby the row of numerals passes the window opening 202 in the housing when the drum is rotated relative to the housing by the ratchet tube. The torque spring is arranged in the circumferential space between the ratchet tube and the reset tube and is at its proximal end secured to the spring base member 208 and at its distal end to the ratchet tube, whereby the spring is strained when the ratchet tube is rotated relative to the housing by rotation of the dial member. A ratchet mechanism with a flexible ratchet arm 252 is provided between the ratchet tube and the clutch element, the latter being provided with an inner circumferential teeth structures 242, each tooth providing a ratchet stop such that the ratchet tube is held in the position to which it is rotated by a user via the reset tube when a dose is set. In order to allow a set dose to be reduced a ratchet release mechanism 262 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and a torsion spring of the spring drive is loaded. The dose mechanism is locked to the housing and cannot move. When the push button is pushed down, the dose mechanism is released from the housing and due to the engagement to the dial system, the torsion spring will now rotate back the dial system to the starting point and rotate the dose system along with it.

The central part of the dose mechanism is the piston rod 220, the actual displacement of the plunger being performed by the piston rod. During dose delivery, the piston rod is rotated by the drive element 230 and due to the threaded interaction with the nut element 225 which is fixed to the housing, the piston rod moves forward in the distal direction. Between the rubber piston and the piston rod, the piston washer 227 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 234 which prevent the drive element from rotating clockwise (seen from the push button end). Due to the engagement with the drive element, the piston rod can thus only move forwards. During dose delivery, the drive element rotates anti-clockwise and the ratchet arms 235 provide the user with small clicks due to the engagement with the ratchet teeth 205, e.g. one click per unit of insulin expelled.

Turning to the dial system, the dose is set and reset by turning the dose dial member 280. When turning the dial, the reset tube 260, the EOC member 228, the ratchet tube 250 and the scale drum 270 all turn with it. As the ratchet tube is connected to the distal end of the torque spring 255, the spring is loaded. During dose setting, the arm 252 of the ratchet performs a dial click for each unit dialled due to the interaction with the inner teeth structure 242 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 202.

The ratchet 252, 242 between the ratchet tube and the clutch element 240 prevents the spring from turning back the parts. During resetting, the reset tube moves the ratchet arm 252, thereby releasing the ratchet click by click, one click corresponding to one unit IU of insulin in the described embodiment. More specifically, when the dial member is turned clockwise, the reset tube simply rotates the ratchet tube allowing the arm of the ratchet to freely interact with the teeth structures 242 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the release button 290 is pushed in the distal direction by the user as shown in FIG. 3B. The reset tube 260 decouples from the dial member and subsequently the clutch element 240 disengages the housing splines 204. Now the dial mechanism returns to “zero” together with the drive element 230, this leading to a dose of drug being expelled. It is possible to stop and start a dose at any time by releasing or pushing the push button at any time during drug delivery. A dose of less than 5 IU normally cannot be paused, since the rubber piston is compressed very quickly leading to a compression of the rubber piston and subsequently delivery of insulin when the piston returns to the original dimensions.

The EOC feature prevents the user from setting a larger dose than left in the cartridge. The EOC member 228 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 270 is provided with a distal stop surface adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism. In the following, the position that the dial member assumes after completion of the expelling of a set dose will be referred to as the “zero dose position”.

To prevent accidental over-dosage in case something should fail in the dialing mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “over dose” of drug to be expelled, e.g. 3-5 IU of insulin.

The expelling mechanism further comprises an end-of-dose (EOD) click feature providing a distinct feedback at the end of an expelled dose informing the user that the full amount of drug has been expelled. More specifically, the EOD function is made by the interaction between the spring base and the scale drum. When the scale drum returns to zero, a small click arm 206 on the spring base is forced backwards by the progressing scale drum. Just before “zero” the arm is released and the arm hits a countersunk surface on the scale drum.

The shown mechanism is further provided with a torque limiter in order to protect the mechanism from overload applied by the user via the dose dial member. This feature is provided by the interface between the dose dial member and the reset tube which as described above are rotationally locked to each other. More specifically, the dose dial member is provided with a circumferential inner teeth structure 281 engaging a number of corresponding teeth arranged on a flexible carrier portion 261 of the reset tube. The reset tube teeth are designed to transmit a torque of a given specified maximum size, e.g. 150-300 Nmm, above which the flexible carrier portion and the teeth will bend inwards and make the dose dial member turn without rotating the rest of the dial mechanism. Thus, the mechanism inside the pen cannot be stressed at a higher load than the torque limiter transmits through the teeth.

In FIG. 3C the clutch element, the drive element and the housing (in partial) are shown in the dose setting state, and in FIG. 3D the same components are shown in the expelling state. As appears, the piston rod on which the drive element is arranged and the ratchet tube on which the clutch element is mounted are not shown. To better show the structures provided on the inner surface of the housing FIG. 3E shows a partial clutch element 240 arranged in the housing 201.

The inner surface of the housing 201 comprises a circumferential ring-formed array of axially oriented spline elements 204 protruding into the interior, each having a pointed distal end 209, as well as a circumferential ring-formed array of one-way ratchet teeth 205. The inner surface further comprises a male helical thread 203 adapted to engage the female helical thread 273 on the scale drum 270. A distal circumferential groove is formed to engage and mount the nut element 225. The clutch element 240 comprises an inner circumferential ring-formed array of ratchet teeth 242 adapted to engage the ratchet arm 252 on the ratchet tube 250, and an outer circumferential ring-formed array of axially oriented spline elements 241 adapted to engage the spline elements 204 of the housing as well as the coupling slots in the drive element (see below), each spline having a pointed proximal end 243. The drive element 230 comprises a pair of opposed coupling portions each comprising two proximally extending skirt portions 232 between which an axially extending coupling slot 233 is formed, the slot being adapted to engage a portion of the clutch element spline elements. In this way the engaging surfaces serve to transmit a rotational force and thereby torque from the clutch element to the drive element in the expelling state. The drive element further comprises a pair of opposed circumferentially extending flexible ratchet arms adapted to engage the ring-formed array of one-way ratchet teeth 205. During dose delivery, the drive element rotates anti-clockwise and the ratchet arms 235 also provide the user with small clicks due to the engagement with the ratchet teeth 205, e.g. one click per unit of insulin expelled. In the shown embodiment 24 ratchet teeth are provided corresponding to 15 degrees rotation per unit of insulin. The central bore of the drive element comprises two opposed protrusions 231 adapted to engage with the axially oriented grooves on the piston rod.

In the dose setting state shown in FIG. 3C the spline elements 241 of the clutch element are in engagement with the spline elements 204 of the housing thereby rotationally locking the clutch element relative to the housing. As can be seen from FIG. 3C a group of clutch spline elements are received in the corresponding coupling slot with a slight rotational play. In the expelling state shown in FIG. 3D the spline elements 241 of the clutch element are moved distally out of engagement with the spline elements 204 of the housing thereby allowing rotation of the clutch element relative to the housing. As can be seen from FIG. 3D the group of clutch spline elements are now received in the corresponding coupling slot without rotational play.

FIG. 3C shows the clutch element 240 showing the above-described inner circumferential ring-formed array of ratchet teeth 242 and the outer circumferential ring-formed array of axially oriented spline elements 241. As appears, the spline elements are not arranged equidistantly on the ring but in groups, the groups comprising two opposed coupling groups 245 serving as the coupling means engaging the coupling slots 233. Whereas thus only some of the spline elements serve as coupling means between the clutch element and the drive element they all serve as coupling means between the clutch element and the housing splines 204.

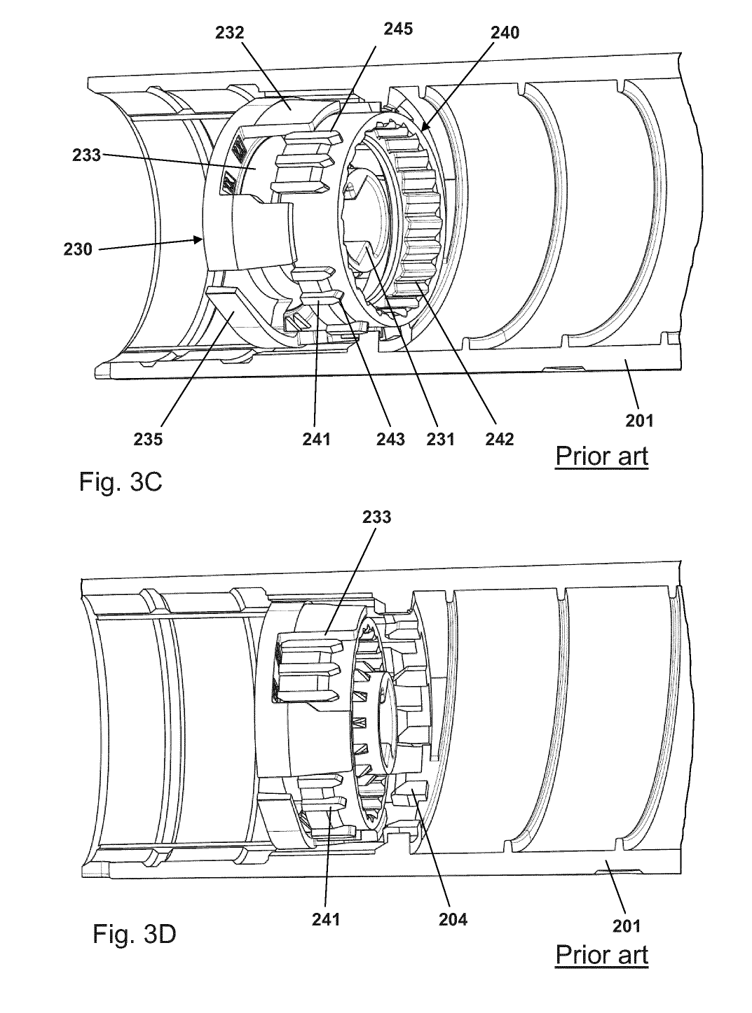

Turning to FIGS. 4A and 4B an exemplary first embodiment of an auto-injection device 100 according to the invention is shown. The auto-injection device 100 defines a housing 101 which accommodates an expelling mechanism comprising a spring-drive. The operating principle of the expelling mechanism may be designed in accordance with the prior art expelling mechanism described in connection with FIGS. 1 through 3E. However, in other embodiments, alternative spring-driven expelling mechanisms having other operating principles may be employed. As a non-limiting example, the operating principles of the auto-injection devices disclosed in WO 2008/116766 may be utilized instead.

FIG. 4B shows the auto-injection device 100 in its storage state, i.e. before the device has been put in use or, alternatively, subsequent to the complete expelling of a set dose. An release button 190 is arranged to protrude from the proximal end of the housing. In the shown embodiment the release button 190 serves as an activation or trigger button 190 similarly to the device shown in FIGS. 1-3. The release button 190 is axially movable relative to the housing between a pushed in position (i.e. an activated position) and an extended position (i.e. an activateable position). In the shown embodiment, the release button 190 is rotationally fixed with respect to the housing by means of a series of splines 194 that engages axial tracks formed internally in housing 101.

A dose dial member 180 serves as a user operable member that may be rotated in order to set a dose. Also, in this embodiment a generally tubular shaped scale drum 170 similarly to the device shown in FIGS. 1-3 serves as a dose setting device in accordance with the present invention. The scale drum 170 is threadedly engaged with the housing 101 to form a multi-turn dose adjuster. Further, the scale drum includes axial tracks that engage coupling elements formed in the dose dial member 180 so that rotation of the dose dial member 180 leads to rotation of the scale drum. The engagement nevertheless allows for relative axial movements there between. A series of dose indications disposed along a helical path on scale drum 170 is observable through window 102 as the scale drum is rotated relative to the housing. A latch function couples the release button 190 with the scale drum 170 to retain the release button in the activated position as long as the scale drum 170 assumes its zero dose position (cf. the discussion in connection with FIGS. 1-3, corresponding to the end of dose position).

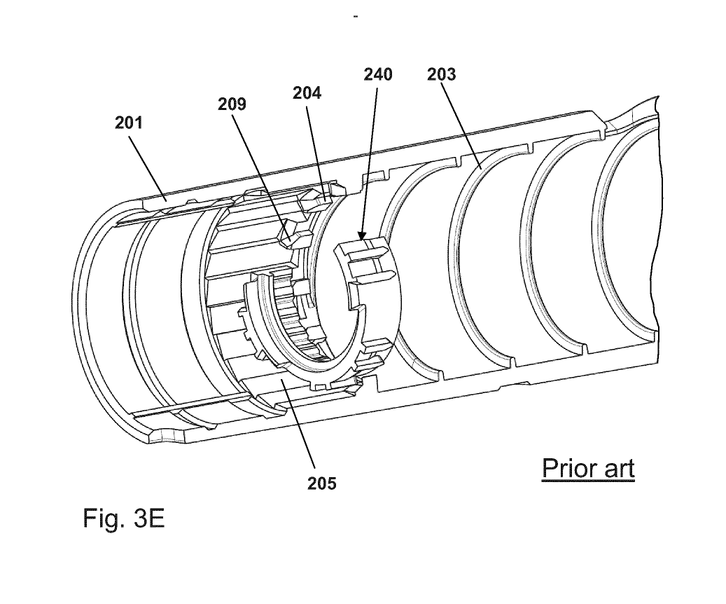

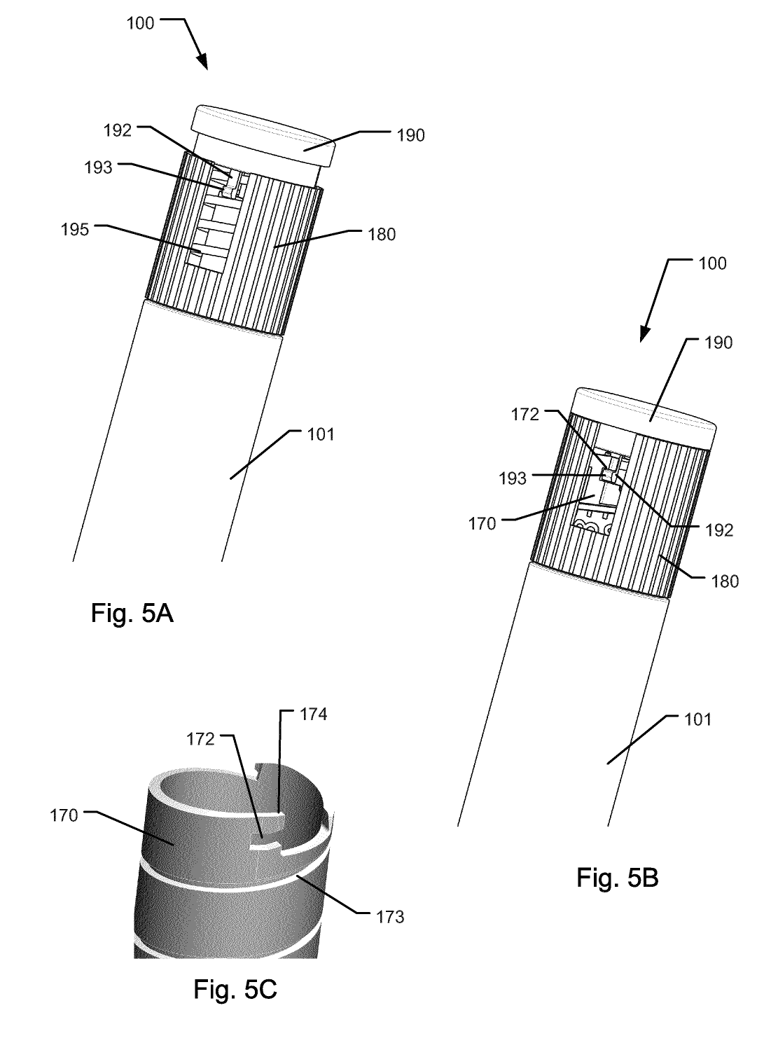

FIG. 4A shows the device 100 in a state where the dose dial member 180 has been rotationally operated to set a dose. The scale drum has been rotated accordingly and moved axially in accordance with the threaded engagement with the housing. The reading of the set dose is visibly observable in window 102. The said latch function is released as the scale drum is moved away from its zero dose position. A release spring 195 (see FIGS. 5A and 7) is accommodated within housing and configured to create a biasing force for urging the release button 190 towards the activateable position. Hence, as the latch is released by dialing away from the zero dose position the release button 190 is automatically forced into its activateable position.

In accordance with the operating principle discussed in connection with FIGS. 1-3, after a dose has been set, the release button 190 may be operated by pushing the release button to its activated position. This will trigger the expelling operation to expel a dose of drug corresponding to the size of the set dose. As the set dose is being expelled the scale drum 170 rotates back to its zero dose position which defines the end of dose state. This state corresponds to the state shown in FIG. 4B. As the scale drum reaches its zero dose position and the user releases the force exerted on the release button 190, the release button stays in the activated position due to the latch function. For the user, this will add to the confidence that the expelling of the set dose has reached its end. At the same time, the pushed in position of release button 190 serves to indicate that the dose dial member 180 needs to be operated for setting a dose before a new injection can be performed.

FIG. 5A shows a partly cut perspective view of the auto-injection device 100 of FIG. 4A in a state where a dose has been dialed up whereas FIG. 5B shows a corresponding view of the device 100 subsequent to the expelling of a full dose. In FIG. 5B the scale drum 170 assumes its zero dose position. FIG. 5B the scale drum 170 is visible in its zero dose position. Further a latch arm 192 is observable.

As best viewed in FIG. 5C the scale drum 170 includes a track or cut-out 172 arranged at the proximal end face of the scale drum. In the shown embodiment the cut-out is generally L-shaped. The track or cut-out 172 is configured for engagement with a latch arm 192. Latch arm 192 is formed as a hook which is mounted relative to release button 190 so that relative axial movement is prevented between release button 190 and latch arm 192. Latch arm 192 includes an enlarged head 193 defining a latch geometry formed to be received in cut-out 172. The cut-out 172 defines a circumferentially extending free end 174 of scale drum 170 that serves as a cooperating retaining geometry adapted to retain the enlarged head 193 against proximal movement.

In FIG. 5B the enlarged head 193 of latch arm 192 is inserted into the L-shaped cut-out 172 as the scale drum rotates towards its zero dose position. Upon reaching its zero dose position, the enlarged head 193 is axially retained relative to the scale drum 170 and hence the release button 190 is retained in its activated position against the force exerted by release spring 195.

Because the scale drum moves axially as it is rotated, there will only be one particular dose setting position, i.e. the zero dose position, where the enlarged head 193 of the latch arm 192 will engage the L-shaped cut-out of the scale drum 170 and remain retained in the activated position. Hence, when the scale drum assumes any other position than its zero dose position the enlarged head 193 will be axially separated from the L-shaped cut-out and the free end 174. Therefore, the release button 190 will constantly be urged by the release spring 195 towards its activateable position provided that no external force is exerted in the distal direction on release button 190 to attempt triggering the device.

In the shown device 100 the enlarged head 193 exhibits an inclined radially outwards facing surface adapted to slide against an radially inwards facing surface of free end 174 of scale drum 170. The latch arm 192 may be formed of flexible material so that the latch arm will be able to flex radially inwards if the enlarged head 193 is moved from a proximal position to a distal position while the scale drum assumes its zero dose position. Such situation might occur if a user by mistake dials up a dose followed by immediately dialing back the scale drum to its zero dose position without triggering an expelling procedure. This will typically leave the activation button in its activateable position. But the flexible latch arm allows the latch arm to flex radially inwards as the enlarged head 193 is forced axially in the distal direction past the free end 174 of scale drum 170 and further distally so that enlarged head 193 is inserted into the L-shaped cut-out 172. Hence, when the scale drum 170 assumes its zero dose position, the release button 190 may be pushed from its activateable position into its activated position simply by manually forcing the activation button into its activated position by applying a distal force on the activation button and the retainment of the activation button in its activated position is re-established.

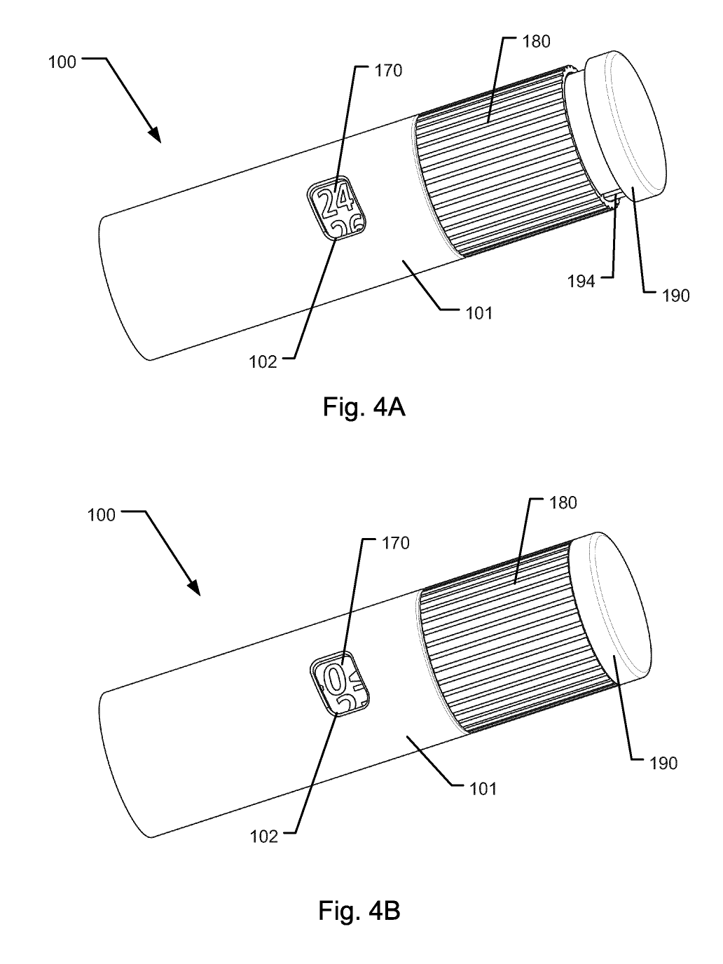

FIGS. 6 and 7 offers additional views of the internal components of the latch function discussed in connection with FIGS. 4-5. FIG. 6 depicts a perspective view of the scale drum 170 and the release button in the retained position where the scale drum assumes its zero dose position and where the release button assumes its activated position. Enlarged head 193 of latch arm 192 is positioned within the L-shaped cut-out 172. FIG. 7 schematically shows a partly cut side view of the auto-injection device shown in FIG. 4A in a state where a dose has been set. In particular, this view reveals the configuration of the release spring 195.

For particular embodiments (not shown), one of the latch geometry and the cooperating retaining geometry may comprise an inclined cam and the other of the latch geometry and the cooperating retaining geometry may comprise a cam follower for engaging the cam. The cam and the cam follower may be formed so that, when no externally applied distal force is applied to the release button, and the release button assumes the activateable position, the cam and cam follower acts to move the release button into its activated position as the dose setting device is returned to the zero dose position. Hence, in such embodiments, the release button will be automatically withdrawn into its activated when the user dials back towards the zero dose position without pushing down the release button.

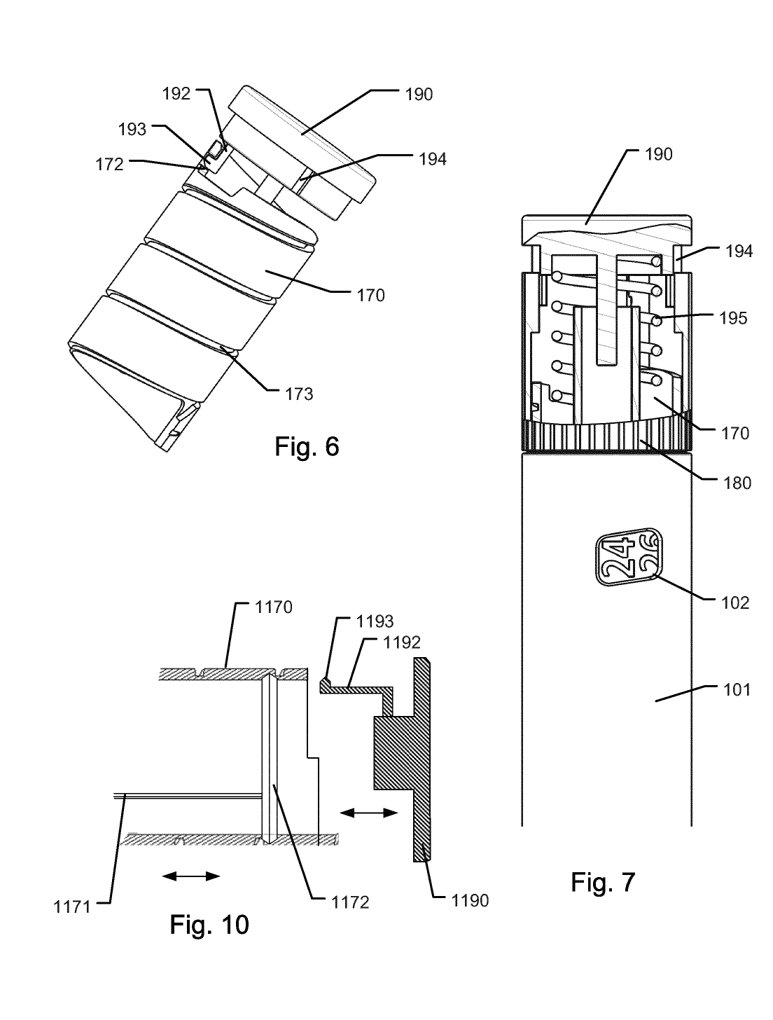

FIGS. 8A and 8B depict to states of a second embodiment according to the invention where the auto-injection device of FIG. 1 has been modified to include the latch function discussed in connection with FIGS. 4-7. FIG. 8A generally corresponds to the state shown in FIG. 3A whereas FIG. 8b generally corresponds to the state shown in FIG. 3B.

A latch arm 1092 generally corresponding to latch arm 192 is incorporated in the proximal end of the device 1000. Latch arm 1092 includes an enlarged head 1093. The latch arm protrudes through an opening in spring base member 1008. Latch arm 1092 is mounted so as to be rotationally fixed relative to housing 1001 but axially movable relative to housing 1001 and spring base member 1008. A proximal end of latch arm 1092 engages reset tube 1060 in such a way that relative axial movements is prevented while relative rotational movement is allowed between reset tube 1060 and latch arm 1092. Hence, the latch arm 1092 is axially fixed relative to release button 1090.

Scale drum 1070 generally corresponds to scale drum 170. Scale drum 1070 includes an L-shaped cut-out 1072 adapted to receive and retain the enlarged head 1093 of latch arm 1092.

In FIG. 8B, which depicts the device 1000 where no dose has been dialed, i.e. where the scale drum is in its zero dose position, the release button 1090 is retained in its activated position. In FIG. 8A, which depicts the device 1000 where a dose has been dialed, the scale drum 1070 has been moved distally in accordance with thread 1003 and the release spring 1095 has thus pushed the release button 1090 into its activateable position, ready for the device to be triggered for expelling the set dose. At the completion of the expelling of the set dose, the release button 1090 and the scale drum 1070 will assume the same respective positions as shown in FIG. 8B and the release button 1090 will be retained in its activated position. However the piston rod (non-referenced) will have moved in the distal direction corresponding to the size of the expelled dose.

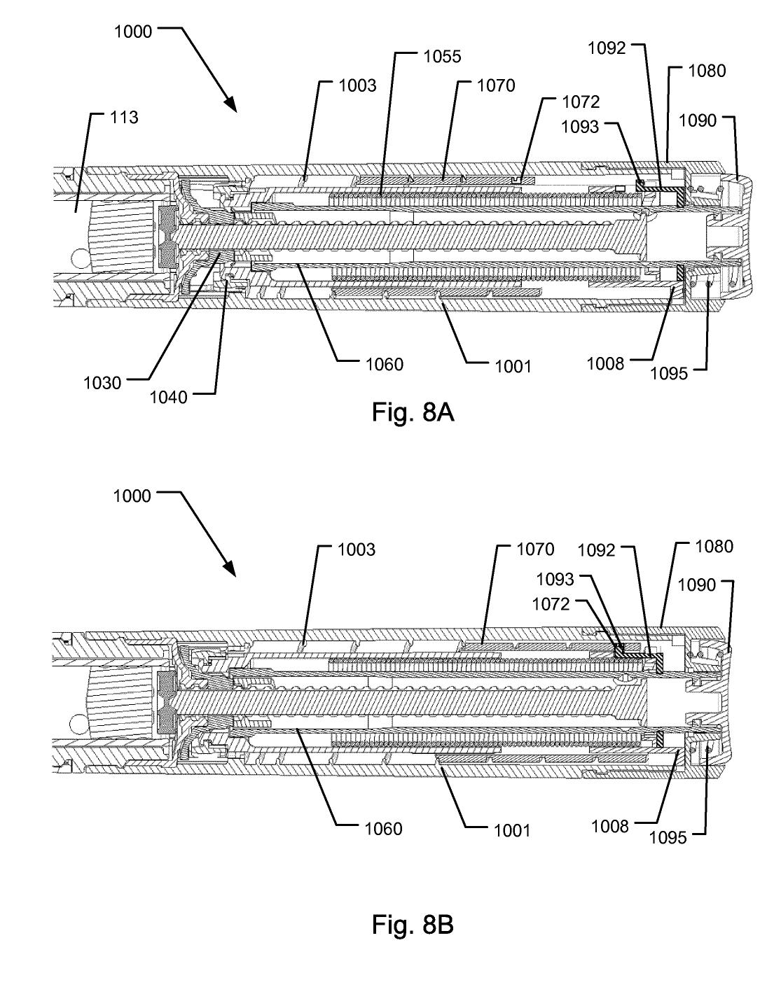

FIGS. 9A and 9B depicts a third embodiment of an auto-injection device 1100 according to the invention where the auto-injection device 1000 of FIGS. 8A and 8B has been modified to include a latch function which differs from the latch function described in connection with FIG. 4A through 8B.

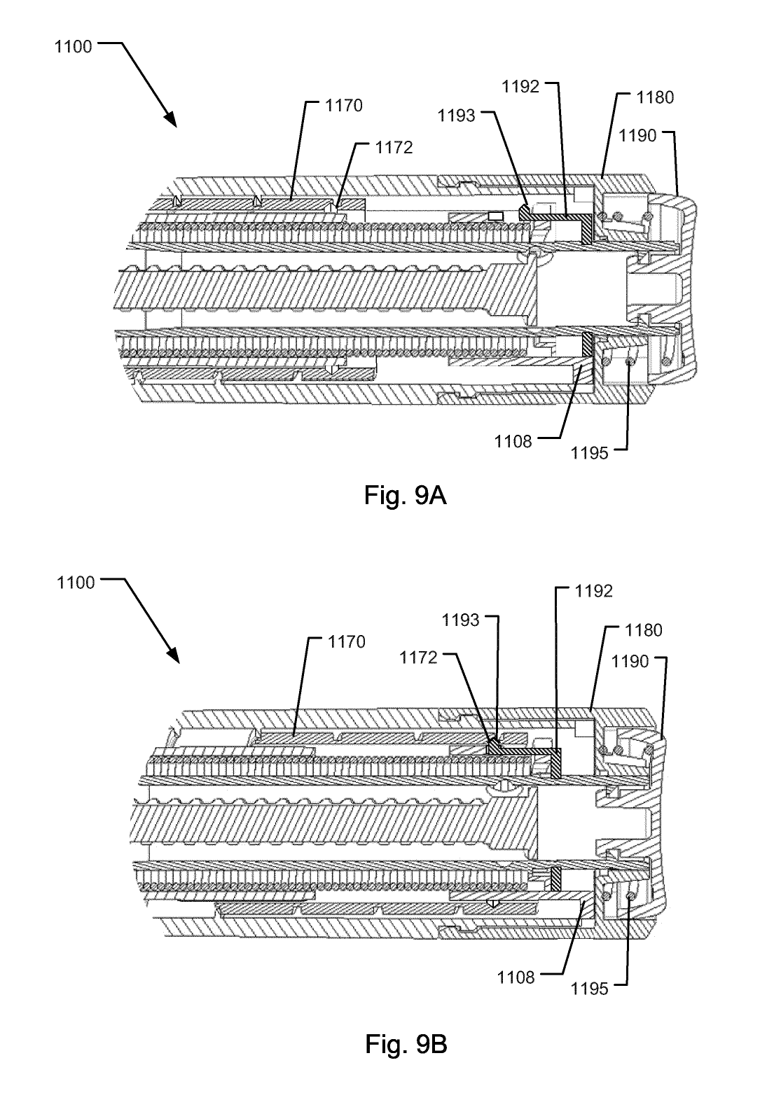

The latch function of the embodiment of the auto-injection device 1100 shown in FIGS. 9A and 9B is further schematically shown in FIG. 10.

Instead of having a latch function where an L-shaped cut-out of the scale drum receives and engages an enlarged head of a latch arm, this latch function relies on a snap function between the release button 1190 and the scale drum 1170. This snap function generally works by providing a snap which is engaged and disengaged simply by axially displacing the scale drum 1170 and the release button 1190 with respect to each other.

In the third embodiment, the latch geometry includes a snap arm 1192 that is able to flex radially inwards. A distal end of snap arm 1192 includes a snap protrusion 1193. In other aspects, the snap arm 1192 is similarly configured as the snap arm 1092 discussed above.

As depicted in FIG. 10, the scale drum 1170 includes an inner circumferential recessed track 1172 adapted to receive the snap protrusion 1193 when the scale drum assumes its zero dose position and when the release button 1190 assumes its activated position. This situation is depicted in FIG. 9B. The snap engagement between snap protrusion 1193 and the recessed track 1172 is sufficiently strong to ensure that the release button 1190 is retained in its activated position.

When the dose dial member 1180 is rotated to dial up a dose, the scale drum 1170 is moved axially in the distal direction in accordance with its threaded engagement with the housing. Hence, the snap protrusion 1193 is forced out of the recessed track 1172 thereby releasing the snap engagement. This forces the snap arm 1192 to flex radially inwards causing the release spring 1195 to urge the release button 1190 into its activateable position as seen in FIG. 9A.

The snap engagement is re-engaged again during expelling of a set dose upon the scale drum 1170 returning to its zero dose position. Alternatively, if a user dials down an initially set dose towards the zero dose position, the release button 1190 may be brought manually back to the activated position by manually pushing the release button into its pushed in position. This also serves to re-engage the snap engagement.

In alternative embodiments the latch geometry according to the second and the third embodiments may include an inclined cam and cam follower generally formed the same way as discussed above in connection with the first embodiment. Such feature would serve to automatically return the release button into its activated position upon the user reducing a set dose by dialing down an initially set dose towards the zero dose setting.

Some preferred embodiments have been shown in the foregoing, but it should be stressed that the invention is not limited to these, but may be embodied in other ways within the subject matter defined in the following claims

1. An auto-injection device for setting and expelling set doses of a liquid drug, comprising:
a housing defining a distal end and a proximal end, the housing adapted for receiving a cartridge so that liquid drug is expellable through a connected needle at the distal end of the housing,
dose setting structure comprising a dose setting device configured for rotation by a helical movement relative to the housing,
an expelling mechanism comprising a spring drive configured for automatically expelling a set dose from a held cartridge upon activation,
an activation button activateable for enabling a set dose to be expelled, and
a release spring providing a force that urges the activation button towards the activateable position,
wherein the auto-injection device defines a latch comprising a latch geometry coupled to the activation button and a cooperating retaining geometry coupled to the dose setting device, the latch being configured to retain the activation button in the activated position against the force of the release spring when the activation button has been activated,
wherein the activation button protrudes from the proximal end of the housing and is axially moveable between an activateable position and an activated position, and the dose setting device is threadedly coupled relative to the housing so that it performs a helical movement away from a zero dose position during setting of a dose and returns by a helical movement to the zero dose position as the set dose is expelled,
wherein:
when the dose setting device assumes its zero dose position, the cooperating retaining geometry assumes a predefined axial position relative to the housing to enable cooperation between the cooperating retaining geometry and the latch geometry as the activation button assumes its activated position, and
when the dose setting device assumes any other position than the zero dose position, the cooperating retaining geometry assumes an axial position which prevents cooperation between the cooperating retaining geometry and the latch geometry for retaining the activation button in its activated position.

2. An auto-injection device as in claim 1, wherein, when the activation button assumes its activated position retained by cooperation between the latch geometry and the cooperating retaining geometry, rotation of the dose setting device away from its zero dose position releases the latch so that the activation button is automatically moved into the activateable position urged by the force for the release spring.

3. An auto-injection device as in claim 1, wherein the latch geometry is disposed on the activation button, and the cooperating retaining geometry is disposed on the dose setting device.

4. An auto-injection device as in claim 1, wherein, when the dose setting device assumes any other position than the zero dose position, the cooperating retaining geometry is axially spaced away from the latch geometry preventing the latch from retaining the activation button in the activated position.

5. An auto-injection device as in claim 1, wherein the dose setting device is threadedly coupled relative to the housing by permanent engagement.

6. An auto-injection device as in claim 1, wherein the dose setting device is rotatable relative to the housing by a rotational movement exceeding 360 degrees.

7. An auto-injection device as in claim 1, wherein the latch geometry comprises a hook member and the cooperating retaining geometry comprises a track arranged to receive the hook member as the dose setting device is rotated towards its zero dose position to thereby retain the hook member against axial displacement.

8. An auto-injection device as in claim 1, wherein the latch geometry comprises a snap member and the cooperating retaining geometry comprises a snap retainer, wherein the snap member is releasably received and retained by the snap retainer when the activation button and the dose setting device assume a pre-defined relative orientation.

9. An auto-injection device as in claim 8, wherein one of the activation button and the dose setting device include a cylindrical surface, wherein the snap retainer comprises a circumferentially extending ledge arranged on the cylindrical surface and wherein the snap arm is configured to engage and snap behind said circumferential extending ledge when the activation button assumes its activated position while the dose setting device assumes its zero dose position.

10. An auto-injection device as in claim 1, wherein the latch geometry comprises a flexible member, the flexible member being able to flex when the activation button is moved from its activateable position into the activated position while the dose setting device assumes its zero dose position, the flexible member serving to allow the latch geometry to engage the cooperating retaining geometry.

11. An auto-injection device as in claim 1, wherein one of the latch geometry and the cooperating retaining geometry comprise an inclined cam and the other of the latch geometry and the cooperating retaining geometry comprise a cam follower for engaging the inclined cam, the inclined cam and the cam follower being so configured that, when no externally applied distal force is applied to the activation button and the activation button assumes the activateable position, the inclined cam and the cam follower acts to move the activation button into its activated position as the dose setting device is returned to the zero dose position.

12. An auto-injection device as in claim 1, wherein the latch geometry and the cooperating retaining geometry are configured for generating a click sound as the latch geometry engages the cooperating retaining geometry upon the dose setting device being returned to its zero dose position.

13. An auto-injection device as in claim 1, wherein the spring drive comprises a drive element configured for distally moving a piston of a held cartridge and a spring element coupled to the drive element, the spring element configured for holding stored energy and, upon activation, releasing energy for urging the drive element to drive the piston for expelling a set dose during an expelling procedure.

14. An auto-injection device as in claim 13, wherein the expelling mechanism further comprises a clutch element coupled to the activation button and the drive element to control release of stored energy of the spring element, and wherein, when the activation button is in the activated position the clutch element enables release of stored energy, and when the activation button is in the activateable position the clutch element prevents release of stored energy.

15. An auto-injection device as in claim 14, wherein, in the course of an expelling procedure, upon removal of an initially applied external distal force on the activation button, the force of the release spring acts to move the activation button to the activateable position to interrupt the expelling procedure.
